(19) United States
(12) Patent Application Publication
(10) Pub. No.: US 2014/0157451 A1
(43) Pub. Date: Jun. 5, 2014

(54) Compositions and Methods for the Generation of Disease Resistant Crops

(71) Applicant: Boyce Thompson Institute for Plant Research, Ithaca, NY (US)

(72) Inventors: Daniel F. Klessig, Dryden, NY (US); Hong-gu Kang, Austin, TX (US); Karl-heinz Kogel, Lollar (DE); Patricia Manosalva, Ithaca, NY (US)

(21) Appl. No.: 14/138,847

(22) Filed: Dec. 23, 2013

Related U.S. Application Data
(63) Continuation-in-part of application No. PCT/US12/43976, filed on Jun. 25, 2012.
(60) Provisional application No. 61/500,343, filed on Jun. 23, 2011.

(51) Int. Cl. C12N 15/82

(52) U.S. Cl. CPC C12N 15/8279
USPC 800/260; 800/279; 800/298; 435/418; 536/23.6; 800/276; 530/370

(57) ABSTRACT

Compositions and methods for creating crops exhibiting enhanced pathogen resistance are disclosed.

This application is a continuation-in-part application of PCT/US2012/043976 filed 25 Jun. 2012 which in turn claims priority to U.S. Provisional Application No. 61/500,343 filed Jun. 23, 2011.

Pursuant to 35 U.S.C. §202(c) it is acknowledged that the U.S. Government has rights in the invention described, which was made in part with funds from the National Science Foundation, Grant Number IOS-0641576.

### FIELD OF THE INVENTION

This invention relates to the fields of transgenic plants and disease resistance. More specifically, the invention provides compositions and methods useful for increasing the resistance of crops to various pathogens.

### BACKGROUND OF THE INVENTION

Several publications and patent documents are cited throughout the specification in order to describe the state of the art to which this invention pertains. Each of these citations is incorporated herein by reference as though set forth in full.

Cereal crops, such as bread wheat (Triticum aestivum L. T. durum L., T. turgidum L.), rice (Oryza sativa L.), maize (Zea mays L.), barley (Hordeumvulgare L.), oat (Avena sativa L.), rye (Secalecereale L.), sorghum (Sorghum bicolor L.), pearl millet (Pennisetumglaucum L.), and Triticum compactum are grasses that belong to the family Poaceae of monocot plants. Many dicot plants are also major or very important crops such as potato (Solanumtuberosum L.), tomato (Solanum lycopersicum L.), soybean (Glycine max L.), sugar beet (Beta vulgaris L.), oilseed rape, (Brassica napus L.), Hop (Humulus lupulus L.), sweet potato (Ipomoea batatas L.), eggplant (Solanum melongena L.), onions (Allium cepa L.), pepper (Capsicum annuum L.), tobacco (Nicotiana tabacum L.), strawberries (Fragaria x ananassa L.), carrots (Daucus carota subsp. sativus L.), and grape (Vita vinifera L.). They have been used for human consumption since the Neolithic age, some 10,000 years ago, and now account for the vast majority of the world food supply (Borlaug, 1998). Domestication and improvement of these crops have mainly been obtained by conventional breeding, and in a few cases, by interspecific and intergeneric hybridizations. During the past century, wide hybridization has been extensively used to develop numerous cultivars with improved agronomic performance, pest tolerance and high yields.

Biotechnology, which includes cell and molecular biology techniques, was developed in the early 1980's. Biotechnology is a powerful tool to increase the understanding of plant growth and development. Recombinant DNA techniques have also provided plant breeders with a vast collection of genes from plants, animals and microbes, some of which are useful for crop improvement. Due to the worldwide predominance of monocot cereal grains in the human diet, cereal crops are the prime targets for improvement by genetic engineering. In contrast, to dictos, including important crops such as tomato and tobacco, which are relatively easy to transform, many studies revealed that transformation of monocot cereals was problematic; in general, monocot cells and tissues were relatively recalcitrant to in vitro regeneration, and did not respond to Agrobacterium-mediated transformation. As a consequence, the first transgenic cereals were not produced until the end of the 1980's, about half a decade after the first transgenic tobacco plants were reported. At present, many of the problems initially encountered during the development of genetic transformation systems for cereals have been overcome, and transgenic rice, maize, wheat and barley are now routinely produced in several laboratories. The increased transformation frequencies for cereals have mainly been the result of: i) systematic screenings of genotypes and explants tissues for suitability in transformation and regeneration systems, ii) reduced soma-clonal variation by shortening the tissue culture period, iii) identification of useful scorable and selectable marker genes, iv) optimization of codon usage and of transcriptional and translational signals to fit the monocot system, v) improvement of direct DNA delivery systems, such as particle bombardment, and vi) adaptation of the Agrobacterium-mediated transformation system to cereals.

Although biotechnological strategies are now widely applicable to monocot, as well as dicot, crops, relevant traits that could be employed in biotechnology approaches in order to improve disease resistance in crops remain to be identified.

### SUMMARY OF THE INVENTION

The present inventors have discovered that modulation of CRT1 and its homologs enhances the resistance of several crops to various pathogens. Thus, the invention is directed to several methods for producing crops which exhibit increased pathogen resistance and the resulting plants and plant parts.

In one embodiment, the method entails introducing a nucleic acid construct encoding at least one RNAi specific for silencing of CRT1 and its related homologs into a plant cell, said at least one RNAi effectively inhibiting CRT1/CRT1 homolog gene expression in said plant cell, plants regenerated from such cells exhibiting increased pathogen resistance when compared to wild type plants lacking said RNAi. Both constitutive and inducible promoters can be employed to control expression of the RNAi. In a preferred embodiment the promoter is an inducible promoter which is induced upon infection with a pathogen. Nucleic acids encoding the RNAi disclosed herein in plant expression vectors are also within the scope of the invention.

In another embodiment, a method for producing crops exhibiting increased pathogen resistance using the TILLNG method is provided. An exemplary method comprises treating plant seeds with an effective amount of an agent effective to introduce mutations into the plant genome and screening the progeny plants for the presence of lesions in the CRT1 gene, the lesions resulting in reduced production of functional CRT1 protein. These CRT1 defective plants are then tested for enhanced resistance to pathogens compared to untreated plants.

In yet another approach, the Ac/Ds transposable element system or a similar transposon system is utilized to increase pathogen resistance in monocots. An exemplary method comprises crossing by breeding, a plant, comprising cells which harbor the Ds transposon elements in their genomes near the CRT1 gene or homologs thereof, to a plant carrying an Ac element which encodes an active transposase, the transposase, catalyzing in the progeny plants, transposition of the Ds element into the surrounding DNA including the CRT1/CRT1 homolog gene. Plants so treated are then screened for the presence of lesions in the CRT1 gene(s), the lesions being correlated with reduced production of functional CRT1/CRT1 homolog protein. These CRT1 defective plants are then tested for enhanced resistance to pathogens compared to untreated plants.

In an alternative embodiment, the method entails introducing a nucleic acid construct encoding CRT1 or its related homologs into a plant, said construct effectively expressing the nucleic acid (gene) at a higher level than that of the endogenous gene (i.e. over expression) or in tissue in which the endogenous gene is not expressed (i.e. ectopic expression), plants generated from such cells exhibiting increased pathogen resistance when compared to wild type plants lacking said nucleic acid construct. Both constitutive and inducible promoters can be employed to control expression of the CRT1 encoding nucleic acid. In a preferred embodiment the promoter is an inducible promoter which is induced upon infection with a pathogen. Nucleic acid constructs encoding the proteins disclosed herein in plant expression vectors, are also within the scope of the invention.

In yet another aspect of the invention, chimeric CRT1 encoding nucleic acids are provided wherein domains from different CRT1 molecules from different species are swapped, thereby altering disease resistance in plants where such chimeric molecules are expressed. Plants expressing such chimeric CRT1 proteins are also provided.

Plants, progeny and seed produced by any of the aforementioned methods are also within the scope of the invention. In a preferred embodiment, the plant is selected from the group consisting of maize, rice, wheat, barley, rye, oats, sorghum, potato, tomato, soybean, pepper, sweet potato, eggplant, onion, carrot, tobacco, strawberry, and grape.

### BRIEF DESCRIPTION OF THE DRAWINGS

FIG. 1: Molecular Phylogenetic analysis of the CRT1 family in plants. The evolutionary history was inferred using the Neighbor-Joining method using a conserved region of the CRT protein sequences from several monocot and dicot plant species: Zm (Zea mays), Os (Oryza sativa), Hv (Hordeumvulgare), At (Arabidopsis thaliana), Sl (Solanumlycopersycum), St (Solanumtuberosum), Nb (Nicotiana benthamiana), Vv (Vitisvinifera), and Gm Glycine max). The bootstrap consensus tree inferred from 1000 replicates is taken to represent the evolutionary history of the taxa analyzed. Branches corresponding to partitions reproduced in less than 50% bootstrap replicates are collapsed. The percentage of replicate trees in which the associated taxa clustered together in the bootstrap test (1000 replicates) are shown next to the branches. The tree is drawn to scale, with branch lengths in the same units as those of the evolutionary distances used to infer the phylogenetic tree. The evolutionary distances were computed using the Poisson correction method and are in the units of the number of amino acid substitutions per site. Phylogenetic analyses were conducted in MEGA4 [1].

FIG. 2A provides the DNA Sequence (SEQ ID NO: 5) and FIG. 2B provides the protein sequence (SEQ ID NO: 6) of clone HvCRT1. Using a barley cv. Golden Promise cDNA and primers deduced from public EST sequence information a full length clone of HvCRT1-a was obtained. Primers SmaI-5HvCRT1-492 5′-CCCGGGAAACCCTAACCTTCCAATGC-3′(SEQ ID NO: 1) and HindIII-3HvCRT1-492 5′-AAGCTTTCACATGTATGGGAGCTGCTG-3′ (SEQ ID NO: 2) were used to amplify the ORF which was subsequently ligated into p35S-BM (DNA Cloning Service, Hamburg, Germany) using SmaI and HindIII.

FIG. 3: Plasmid p35S-HvCRT1 is shown.

FIG. 4: HvCRT1 negatively regulated MLA12-mediated resistance in barley to the powdery mildew fungus Blumeriagraminis f. sp. hordei: overexpression of HvCRT1 induces susceptibility. Barley cv. Sultan5 carrying resistance gene Mla12 was transiently transformed by co-bombardment with p35S::HvCRT1, p35S::Mlo(hyper-susceptible) and pGY1-GFP. Average penetration efficiency of Bgh-A6 on cv. Sultan 5 was assessed in 3 experiments at 24 h after bombardment and one experiment 4 h after bombardment (shown by black bars). Control: co-bombardment with Mlo and empty vector p35S:BM (shown by grey bars). Statistics: t-test ***p<0.001.

FIG. 5: Plasmid p-AB 355-RNAi ZeBaTA #423-3. Clone #423-3: fragment of HvCRH1, TA cloning of PCR product into p-AB 35S-RNAi ZeBaTA (#407) using HvCRTfwd #996v 5′-GAGACTTGGTGCTGATGCAA-3′(SEQ ID NO: 3) and HvCRTrev #997v 5′-TTTTGACCTTGATCCCGAAG-3′(SEQ ID NO: 4). The sequence of HvCRH1 has the cacc. No. BAJ92329.1. The sequence of the SfiI site is SEQ ID NO: 7.

FIG. 6: HvCRH1 negatively regulated MLA12-mediated resistance in barley to the powdery mildew fungus Blumeriagraminis f. sp. hordei; silencing of HvCRH1/HvCRT1 enhances resistance. Barley plants carrying MLA12 were particle bombarded with an RNAi construct targeting HvCRH1/HvCRT1, followed by the inoculation with the Bgh-A6 fungus 24 h later (black). RNAi empty vector was used as a control (gray). Shown is the % of successful infection sites, as indicated by formation of haustoria or their initials, among all host cells which have been both i) transformed with the silencing constructs and ii) attacked by the fungus. A minimum of 150 sites were evaluated. A GFP reporter gene was used to identify transformed cells.

FIG. 7: Sequence alignment of HvCRH1-RNAi423 and HvCRT1. The sequence of HvCRH1-RNAi423 is SEQ ID NO: 8 and the sequence of HvCRT1-492 is SEQ ID NO: 9.

FIG. 8: RNAi-mediated knockdown of CRH1 renders barley plants (cv. Golden Promise) more resistant to the biotrophic powdery mildew fungus. The number of colonies was measured on 10-day-old Empty vector (control) and HvCRH1-RNAi plants, after the second leaf was detached and subsequently inoculated with conidia of Blumeria graminis f. sp. hordei (race Bgh-A6). L11, L40, and L55 represent plant batches from independent knockdown lines. The mean of 20 plants per data point is presented. Note that cv Golden Promise does not contain an R gene matching the race Bgh-A6. (±SE; Student's t-test p<0.01**, p<0.001***).

FIG. 9: HvCRH6 silencing increased resistance in barley against powdery mildew fungus. Barley leaves (cv. Sultan5) carrying resistance gene Mla12 were co-bombarded with a 35S promoter-driven RNAi construct targeting HvCRH6, along with a construct containing the 35S promoter-driven GFP reporter gene. Leaves were inoculated 24 h later with Blumeria graminis f. sp. Hordei (Bgh-A6) which contains AvrMla12. Average penetration efficiency of Bgh-A6 sporelings was assessed in 4 experiments at 48 h after inoculation. Control: co-bombardment with Mlo, GFP, and an RNAi construct targeting uida (GUS). Between 58 and 137 interaction sites per transformant were evaluated in each experiment. The GFP reporter gene was used to identify transformed cells. The Mlo gene was used to enhance overall penetration rates. Note that over-expression of HvCRH6 resulted in reduced resistance comparable with the CRT1 phenotype (data not shown). Statistics: t-test* significant at p<0.05.

FIG. 10. Disease severity in Fusariumgraminearum infected seedlings of CRH1 knockdown line L55 is reduced compared with the control (Empty vector plants). Seven-day-old seedlings were scored for leaf, coleoptile and root necrosis on a scale of 0-4 (resistant to susceptible). Presented here is the average score of 12 seedlings assessed for these phenotypes. Note that leaf infections are especially reduced as those are particularly critical for the emergence of head blight disease.

FIG. 11. Silencing HvCRH1 enhanced barley growth in Fusarium graminearum infected seedlings as assessed by increase in shoot and root length. Shoot and root lengths of 7-day-old seedlings were measured to assess possible growth retardation caused by Fusarium infection. All measurements were performed using the java based image processing program ImageJ. Mean organ length of 12 seedlings±SE is presented. (Student's t-test p<0.001***).

FIG. 12. Overexpression (Oex) of CRT1 in barley (cv. Golden Promise) lowers resistance to powdery mildew. The number of colonies was measured in 10-day-old control (Empty vector) and HvCRT1-OE plants after the second leaf was detached and inoculated with conidia of Blumeria graminis f. sp. hordei (Bgh-A6). L5, L8, and L13 represent independent transformants. The mean of 25 plants per data point is presented. (±SE; Student's t-test p<0.01**, p<0.001***).

FIG. 13. Average CRT1 transcript levels in T1 plants of three different transformants L5, L8 and L13. The levels of HvCRT1 transcripts were normalized to HvUbiquitin. Each column represents 10 plants. Level of HvCRT1 over expression (Oex) correlated with the reduction in resistance to powdery mildew shown in FIG. 12.

FIG. 14 shows that silencing SlCRT1 in tomato enhances resistance to the oomycete pathogen Phytophthora infestans. SlCRT1-silenced transgenic M82 tomato plants (RNAi) were inoculated with a sporangia suspension (4000 esporangia/ml) of a US-22 isolate (US100041) using a detached leaflet assay. Measurements of the lesion size in cm2 and sporangia number/ml counting were done at 5 and 7 days post inoculation respectively. Asterisks indicate statistically significant differences (*P<0.05, student t test) between the disease symptoms (blighted area and sporangia numbers) of empty vector plants (EV) to those in the RNAi transgenic plants.

FIGS. 15A and 15B show that SlCRT1 in tomato increases susceptibility to the oomycete pathogen Phytophthora infestans. M82 tomato independent transgenic plants overexpressing SlCRT1 under estradiol inducible promoter (OE) were inoculated with a sporangia suspension (4000 esporangia/ml) of two isolates of the pathogen: FIG. 15A) US-22 (US100041) and FIG. 15B) US-11 (US050007) using a detached leaflet assay. Measurements of the lesion size in cm2 and sporangia number/ml counting were done at 5 or 6 days post inoculation with US-22 and US-11 respectively. Asterisks indicate statistically significant differences (*P<0.0001, student t test) between the disease symptoms (blighted area and sporangia numbers) of empty vector plants (EV) to those in OE transgenic plants. These experiments were done twice with similar results.

FIGS. 16A and 16B show that StCRT1 in potato increases susceptibility to the oomycete pathogen Phytophthora infestans. FIG. 16A: StCRT1-silenced transgenic Desiree potato plants (RNAi) were inoculated with a sporangia suspension (4000 esporangia/ml) of a US-11 (US050007), US-22 (US100041), and US-8 (US100021) isolates of P. infestans using a detached leaflet assay. Measurements of the lesion size in cm2 were done at 6 dpi with US-11 and at 5 dpi with the other two isolates. Asterisks indicate statistically significant differences (*P<0.05, student t test) between the disease symptoms (blighted area) of empty vector plants (EV) to those in the RNAi transgenic plants. FIG. 16B: Spray inoculation of EV and StCRT1-silenced potato plants was done to confirm the results from the detached leaflet assay using a sporangia suspension (4000 esporangia/ml) of the US-22 isolate. Percentage of disease was done at 5 (data not shown) and at 6 dpi. These experiments were done twice with similar results.

FIGS. 17A and 17B show that overexpressing StCRT1 in potato enhances resistance to the oomycete pathogen Phytophthora infestans. FIG. 17A: Potato (Desiree) independent transgenic plants overexpressing StCRT1 under estradiol inducible promoter (OE) were inoculated with a sporangia suspension (4000 esporangia/ml) of a US-22 (US100041) isolate of P. infestans using a detached leaflet assay. Measurements of the lesion size in cm2 and sporangia number/ml counting were done at 4 or 7 days post inoculation respectively. Asterisks indicate statistically significant differences (*P<0.05, student t test) between the disease symptoms (blighted area and sporangia numbers) of empty vector plants (EV) to those in OE transgenic plants. FIG. 17B: Spray inoculation of EV and OE transgenic potato plants was done to confirm the results from the detached leaflet assay using a sporangia suspension (4000 esporangia/ml) of the US-22 isolate. Percentage of disease was done at 5 (data not shown) and at 6 dpi.

FIGS. 18A and 18B show chimeric CRT1 molecules which alter disease resistance phenotypes. Schematic diagrams of the domain structure of CRT1 are provided. FIG. 18A: A first example of domain swapping between tomato and potato. FIG. 18B: A second example of domain swapping between cereal CRT1 and barley CRT1.

### DETAILED DESCRIPTION OF THE INVENTION

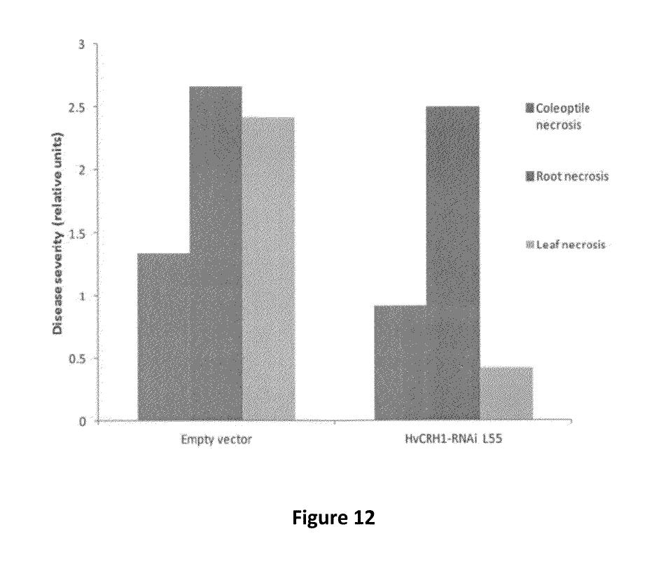

In accordance with the invention, a trait (gene) has been identified that should significantly improve disease resistance in crops, including cereals, and thus could be employed in future breeding strategies to generate high performance crops, including cereal crops, cultivars for agricultural use in conventional, organic, and GMO-based production systems. In previous work, the CRT1 gene family was shown to be required for disease resistance in Arabidopsis (Kang et al., 2008 & 2010). Inactivating or silencing (via RNAi technology) CRT1 resulted in compromised resistance to viral, bacterial and oomycete pathogens. Unexpectedly, we have now found that modulation of CRT1 or its homologs CRH1 and CRH6 expression in other species such as barley and tomato has the completely opposite effect. This was demonstrated using barley (Hordeumvulgare L.) and the powdery mildew-causing fungus Blumeriagraminis f. sp. hordei and the toxin-producing fungus Fusariumgraminearum, and using tomato (Solanumlycopersicum L.) and the late blight-causing oomycetes Phytophthora infestans. In contrast, over expression of CRT1 in potato resulted in enhanced resistance to Phytophthora infestans. (Note over expression of CRT1 in Arabidopsis did not enhance resistance.) These results indicate that modulation of expression or function of CRT family members can enhance resistance to pathogens in a species-specific manner, where silencing or inactivation CRT family members enhances resistance in plants such as barley and tomato, while resistance in other species such as potato can be enhanced by their over expression. This species-specific modulation enhances resistance in both dicot (e.g. potato and tomato) and monocot (barley) crops to several different types of major pathogens including the fungi Blumeria graminis and Fusarium graminearum and the oomycetes Phytophthora infestans, arguably one of the most virulent and devastating plant pathogens, which caused the Great Irish Potato Famine of the 1840s and remains a major threat to food security worldwide today. This species-specific modulation can enhance basal resistance in crop cultivars that do not carry an appropriate disease resistance (R) gene to the pathogen, as we demonstrated in barley to Fusarium and potato and tomato to Phythphthora. It can also enhance even R gene-mediated resistance, as demonstrated in barley containing the MLA12 R gene to Blumeria graminis. Moreover, this enhanced resistance will increase plant growth/production as demonstrated in barley after infection with Fusarium (FIG. 12).

The Arabidopsis CRT1 family has seven members divided into three subfamilies. Subfamily I consists of CRT1, as well as CRH1 and CRH2, which are highly homologous (70-80% amino acid identity) to CRT1 due to tandem triplication. They are also functionally redundant with CRT1. Subfamily II contains three members CRH3—CRH5, which are more distantly related to CRT1 (45-50% amino acid identity). Subfamily III contains CRH6 and is most distantly related to CRT1. Interestingly, CRT1 dimerizes (or oligomerizes) not only with itself, but also with CRH3 and CRH6, although less readily, suggesting the possibility that intra family interactions between CRT1 family members may play a role in modulating resistance. All crop species contain CRT1 and most contain CRH homologs from the other two subfamilies/clades. Enhancement or resistance by modulating of their expression has been shown in barley for CRT1 and two of its homologs CRH1 and the most distantly related CRH6.

### I. DEFINITIONS

The phrase “CRT1 function” is used herein to refer to any CRT1 activity, including without limitation expression levels of CRT1, CRT1 enzymatic activity, and/or modulation of disease resistance or immune signaling. CRT1 is a member of the GHKL ATPase/kinase superfamily and interacts with various resistance proteins from different structural classes, and this interaction is often disrupted when these resistance proteins are activated.

A “CRT1 homolog” is any protein or DNA encoding the same which has similar structural properties (such as sequence identity and folding) to CRT1.

The term “pathogen-inoculated” refers to the inoculation of a plant with a pathogen.

The phrase “disease defense response” refers to a change in metabolism, biosynthetic activity or gene expression that enhances a plant's ability to suppress the replication and spread of a microbial pathogen (i.e., to resist the microbial pathogen). Examples of plant disease defense responses include, but are not limited to, production of low molecular weight compounds with antimicrobial activity (referred to as phytoalexins) and induction of expression of defense (or defense-related) genes, whose products include, for example, peroxidases, cell wall proteins, proteinase inhibitors, hydrolytic enzymes, pathogenesis-related (PR) proteins and phytoalexin biosynthetic enzymes, such as phenylalanine ammonia lyase and chalcone synthase (Dempsey and Klessig, 1995; Dempsey et al., 1999). Such defense responses appear to be induced in plants by several signal transduction pathways involving secondary defense signaling molecules produced in plants. Certain of these defense response pathways are SA dependent, while others are partially SA dependent and still others are SA independent. Agents that induce disease defense responses in plants include, but are not limited to: (1) microbial pathogens, such as fungi, oomycetes, bacteria and viruses; (2) microbial components and other defense response elicitors, such as proteins and protein fragments, small peptides, β-glucans, elicitins, harpins and oligosaccharides; and (3) secondary defense signaling molecules produced by the plant, such as SA, H₂O₂, ethylene, jasmonates, and nitric oxide.

The terms “defense-related genes” and “defense-related proteins” refer to genes or their encoded proteins whose expression or synthesis is associated with or induced after infection with a pathogen to which the plant is usually resistant.

A “transgenic plant” refers to a plant whose genome has been altered by the introduction of at least one heterologous nucleic acid molecule.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The phrase “Ac/Ds transposable element system” refers to a method of mutagenesis employing a transposon which jumps or inserts into a gene of interest (e.g., CRT1 or homologs thereof) and produces a mutation. The presence of the transposon provides a straightforward means of identifying the mutant allele, relative to chemical mutagenesis methods.

“Ac (activator)” is a transposase which enables a transposon to “jump” into different regions in a targeted plant genome.

“Ds (dissociator)” refers to a transposon which upon transposase action inserts and thereby “marks” chromosomal regions where chromosome breakage occurs (e.g., to alter CRT1 gene expression in a targeted plant).

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90 95% by weight of the given compound. Purity is measured by methods appropriate for the given compound (e.g. chromatographic methods, agarose or polyacrylamide gel electrophoresis, HPLC analysis, and the like).

A “replicon” is any genetic element, for example, a plasmid, cosmid, bacmid, phage or virus, that is capable of replication largely under its own control. A replicon may be either RNA or DNA and may be single or double stranded.

A “vector” is any vehicle to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “promoter region” refers to the 5′ regulatory regions of a gene (e.g., CaMV 35S promoters and/or tetracycline repressor/operator gene promoters).

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion, biolistic delivery, and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

The phrase “double-stranded RNA mediated gene silencing” refers to a process whereby target gene expression is suppressed in a plant cell via the introduction of nucleic acid constructs encoding molecules which form double-stranded RNA structures with target gene encoding mRNA which are then degraded.

The term “co-suppression” refers to a process whereby expression of a gene, which has been transformed into a cell or plant (transgene), causes silencing of the expression of endogenous genes that share sequence identity with the transgene. Silencing of the transgene also occurs.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

“Mature protein” or “mature polypeptide” shall mean a polypeptide possessing the sequence of the polypeptide after any processing events that normally occur to the polypeptide during the course of its genesis, such as proteolytic processing from a polyprotein precursor.

A low molecular weight “peptide analog” shall mean a natural or mutant (mutated) analog of a protein, comprising a linear or discontinuous series of fragments of that protein and which may have one or more amino acids replaced with other amino acids and which has altered, enhanced or diminished biological activity when compared with the parent or nonmutated protein.

The present invention also includes active portions, fragments, derivatives and functional or non-functional mimetics of CRT1-related polypeptides, or proteins of the invention. An “active portion” of such a polypeptide means a peptide that is less than the full length polypeptide, but which retains measurable biological activity.

A “fragment” or “portion” of an CRT1-related polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the CRT1-related polypeptide sequence, antigenic determinants, or epitopes are useful for eliciting immune responses to a portion of the CRT1-related protein amino acid sequence for the effective production of immunospecific anti-CRT1 antibodies.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim, an in the latter instance, impurities ordinarily associated with the specified material(s). The term “consisting essentially of” limits the scope of a claim to the specified elements, steps or materials and those that do not materially affect the basic and novel characteristic(s) of the claimed subject matter.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

A “clone” or “clonal cell population” is a population of cells derived from a single cell or common ancestor by mitosis.

A “cell line” is a clone of a primary cell or cell population that is capable of stable growth in vitro for many generations.

### II. GENERATION OF TRANSGENIC CROPS WITH ENHANCED PATHOGEN RESISTANCE BY MODULATION OF EXPRESSION OF CRT1 FAMILY GENES

The information provided herein enables the production of crops which exhibit enhanced resistance to plant pathogens. In one approach, transgenic barley and other crops will be constructed using the RNA interference (RNAi) vector pLH6000 (DNA Cloning Services, Hamburg, Germany) under which HvCRH1 or HvCRT1 is constitutively expressed under the CaMV 35S promoter. In parallel, an RNAi version of HvCRH1 or HvCRT1 will be placed under control of an pathogen-inducible promoter such as the barley PR-1 promoter, the barley PRb-1 promoter (or any pathogen-inducible promoter with activity in cereals, such as the promoters of barley pathogenesis-related proteins, or the promoter of the Mlo gene), whose expression is rapidly induced upon infection in both infected, local and uninfected, systemic tissues. An exemplary PR-1 promoter is disclosed in U.S. Pat. No. 5,689,044, the entire disclosure of which is incorporated herein by reference.

The TILLING method combines a standard and efficient technique of mutagenesis with a chemical mutagen such as Ethyl methanesulfonate (EMS) with a sensitive DNA screening technique that identifies single base mutations (also called point mutations) in a target gene. EcoTILLING is a method that uses TILLING techniques to look for natural mutations in individuals, usually for population genetics analysis. The TILLING method relies on the formation of heteroduplexes that are formed when multiple alleles (which could be from a heterozygote, or a pool of multiple homozygotes and heterozygotes) are amplified in a PCR, heated, and then slowly cooled. A “bubble” forms at the mismatch of the two DNA strands (the induced mutation in TILLING or the natural mutation in EcoTILLING), which is then cleaved by single stranded nucleases. The products are then separated by size on several different platforms.

The second method is based on the Ac/Ds transposable element system discovered by Barbara McClintock. Insertion of the Ac or Ds element inactivates the gene and its encoded protein. Ac elements encode a functional transposase that enable it, as well as Ds elements, to jump/transpose to other parts of the genome. Ds elements are fragments of an Ac element that cannot on their own jump because they do not encode a functional transposase. However, they can jump via the use of the transposase provided in trans by Ac. Tom Brutnell's group has shown that genes within a 2- to 3-centimorgan region flanking Ds insertions serve as optimal targets for regional mutagenesis (Vollbrecht et al., 2010). Since the genomes of most of the crop cereals, including maize, have been sequenced and since Brutnell's group has developed maize lines with Ds elements distributed around the different chromosomes and different part of the chromosomes, one can select a line which has a Ds near the gene of interest. Crossing Ac into that line facilitates the Ds element to jump into adjacent DNA, including the gene of interest such as CRH1.

In another embodiment, overexpression of the CRT1 gene is induced in a target population of plant cells to increase disease resistance in plants. This elevated expression leads to overproduction of the encoded protein, CRT1 and serves to increase resistance in certain plant species. Overproduction of CRT1 in transgenic plant cells may be assessed at the mRNA or protein level using standard technique known in the art such as RT-PCR. Alternatively, overexpression of CRT1 by this method may facilitate the isolation and characterization of other components involved in the protein-protein complex formation that occurs during the initiation of the disease resistance response pathway in plants. Inasmuch as the sequence encoding CRT1 is known for a variety of plant species, overexpression of the CRT1 encoding nucleic acid is readily achievable in targeted plants species using strong constitutive promoters such as CaMV35S and the like. Alternatively, in cases where inducible expression is preferred, the inducible PR-1 promoter, for example, can be employed. The skilled person in this art area is aware of the many plant vectors and plant gene expression control sequences that are suitable for expression a heterologous gene of interest in a particular plant species.

The aforementioned approaches are suitable for modulating CRT family member expression in targeted plants thereby enhancing pathogen resistance in crops, such as barley, tomato and potato.

The following examples are provided to illustrate certain embodiments of the invention. They are not intended to limit the invention in any way.

### Example I

### CRT1 Gene Silencing Enhances Disease Resistance in Barley

### Isolation of CRT1 Genes in Monocot Cereal Crops:

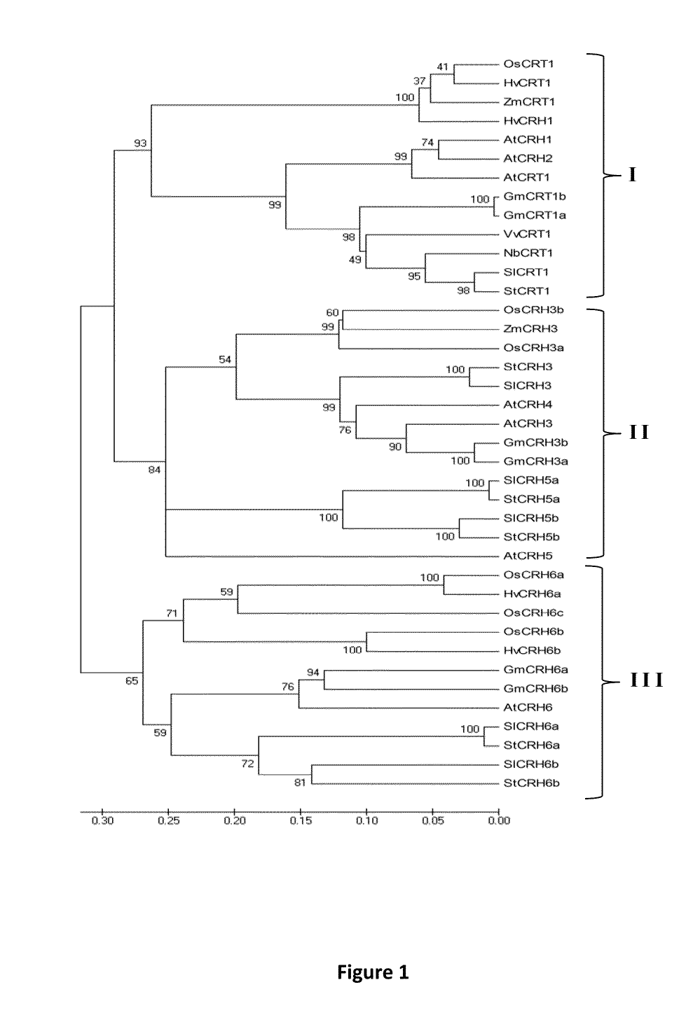

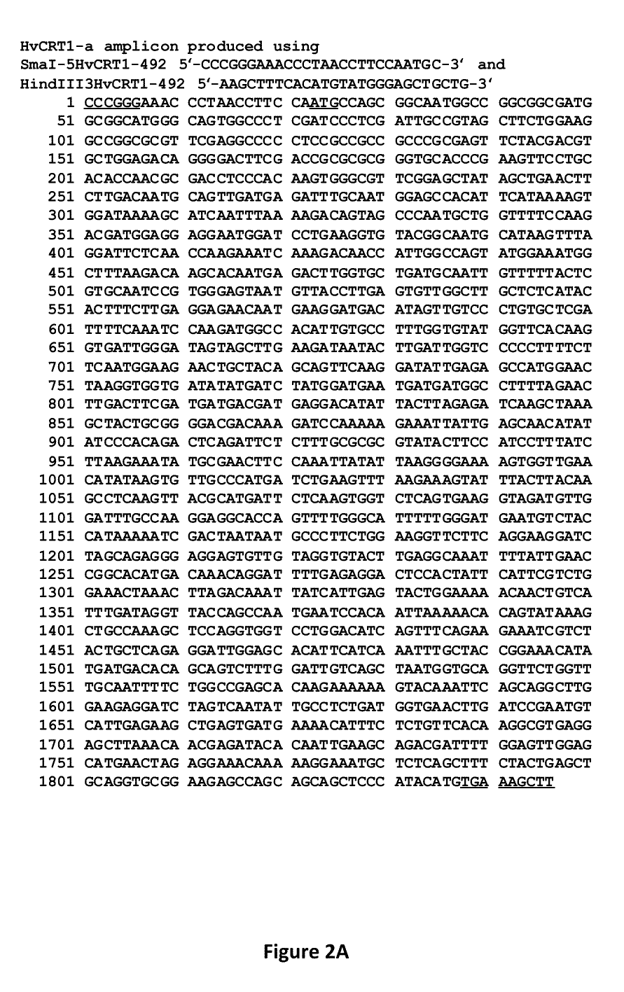

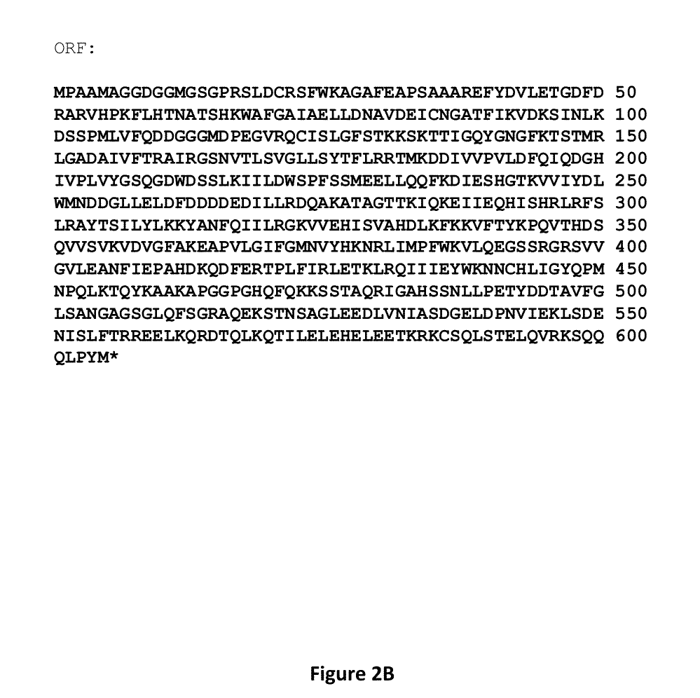

To assess the role of CRT1 genes in resistance to Bgh, two complementary approaches were employed—transient over expression of HvCRT1 or silencing of HvCRH1 or HvCRH6 or one of its homologs. Based on fragmentary data from the emerging barley DNA sequence database, four genes were isolated from the barley cultivar Golden Promise. Two genes have high similarity to AtCRT1 (HvCRT1, HvCRH1) and cluster together with the rice and maize CRT1 homologs, OsCRT1 and ZmCRT1, respectively. Two other genes (HvCRH6a and HvCRH6b) have also been identified which show high similarity to AtCRH6 (FIG. 1). The DNA and protein sequences of the novel clone HvCRT1 are shown in FIGS. 2a and b.

### Assessment of CRT1 Family Gene Function in Plant Responses to Pathogens:

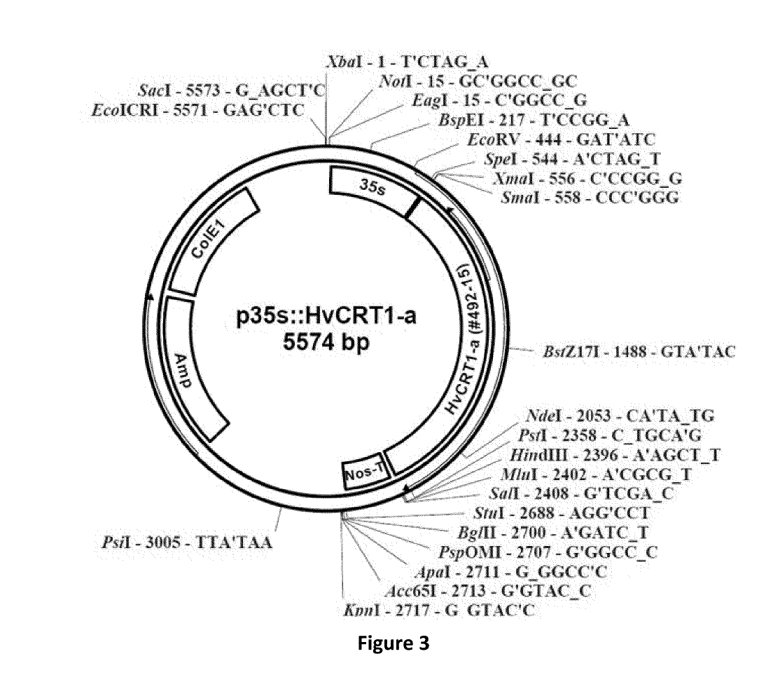

To assess the function of CRT1 and its homologs in a crop plant, we used an established assay for transient genetic transformation in which the test gene (i.e., CRT1 homologs) were bombarded (shot) using a particle gun into epidermal cells of barley leaves prior to infection with the powdery mildew fungus. The method was first described in Schweizer et al. 1999 and 2000. In preparation for shooting, HvCRT1 (see FIG. 2) was ligated into p35S::BM (DNA Cloning Service, Hamburg, Germany) using SmaI and HindIII. The resulting plasmid p35S::HvCRT1 (FIG. 3), containing the HvCRT1 gene under control of the CaMV 35S promoter, was subsequently used in the transient transformation assay. Briefly, barley plants cv. Sultan5, bearing the powdery mildew resistance gene Mla12, were grown in a growth chamber at 18° C. with 60% relative humidity and a photoperiod of 16 h (60 μmol photons m⁻² s⁻¹). For each experiment, sixteen detached 7-day-old first leaves were bombarded using a particle inflow gun (Biorad) with DNA-coated tungsten particles (approximately 310 μg per 1.1 μl particles).

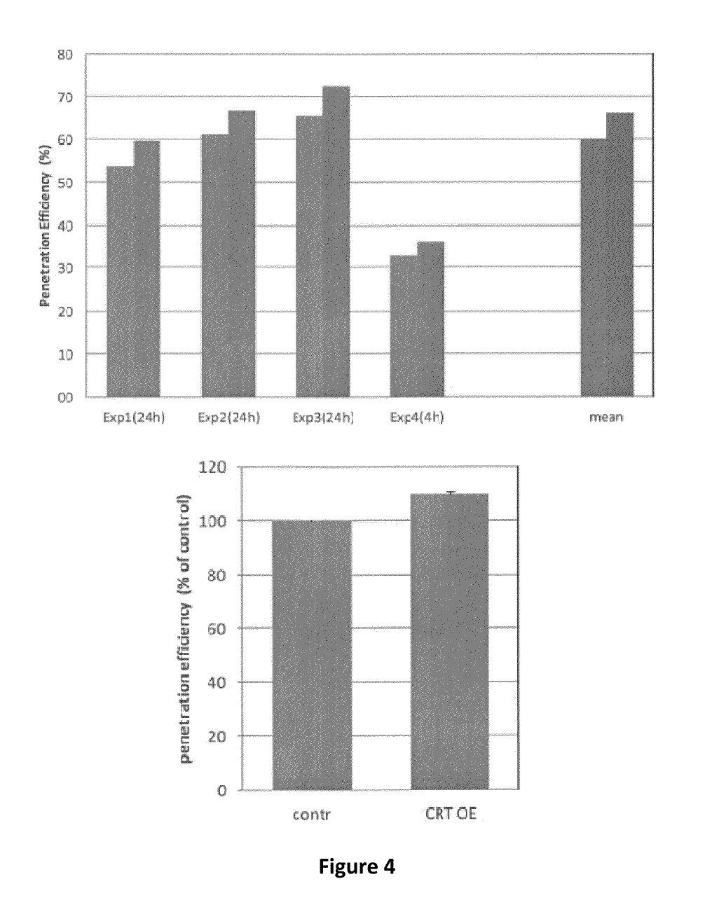

To visualize transformed epidermal cells and to increase susceptibility, two additional plasmids were co-bombarded with p35S::HvCRT1. These included i) plasmid pGY1-GFP (containing a GFP reporter gene to identify those cells hit by gene-coated particles and transiently expressing those genes) and ii) plasmid p35S::Mlo (containing the HvMlo gene that enhances penetration rates of powdery mildew fungi). As a control, the empty vector p35S BM together with pGY1-GFP and p35S::Mlo was used. Four h or 24 h later, leaves were inoculated with conidia of Blumeriagraminis f. sp. hordei race A6 (avirulent on Mla12; the fungal culture is available from the culture collection of the Institute of Phytopathology and Applied Zoology, JLU Giessen, Germany) and 48 h later penetration efficiency was evaluated at single cell level using fluorescence microscopy. FIG. 4 shows the results of four independent experiments in which the frequency of successful penetration by Bgh-A6 (as indicated by formation of mature or immature haustoria) on cells transformed with the three transgene was determined.

Constructs used in the above experiments:
A: p35S-HvCRT1 together with: pGY1-GFP and p35S-HvMlo.
B: Control plasmid (p35S-BM) together with: pGY1-GFP and p35S-HvMlo

The results from these experiments show that over-expression of HvCRT1 significantly enhances the frequency of successful Bgh-A6 penetration strongly suggesting that HvCRT1 suppresses resistance of barley to powdery mildew (FIG. 4). This result argues that CRT1 negatively regulates/affects resistance.

### A Genetic Strategy (Method) to Enhance Disease Resistance:

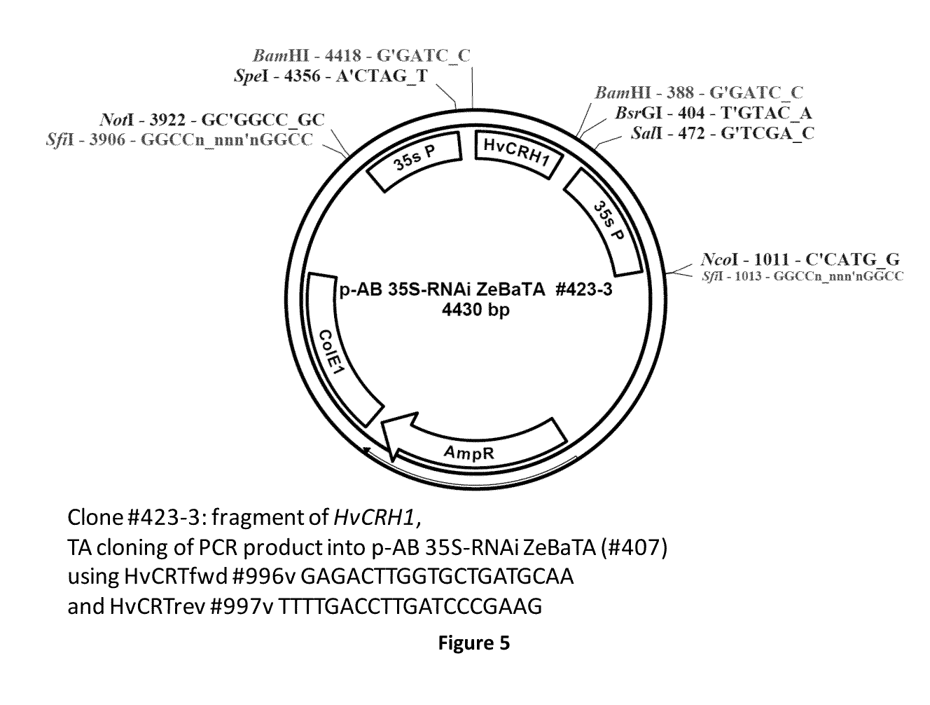

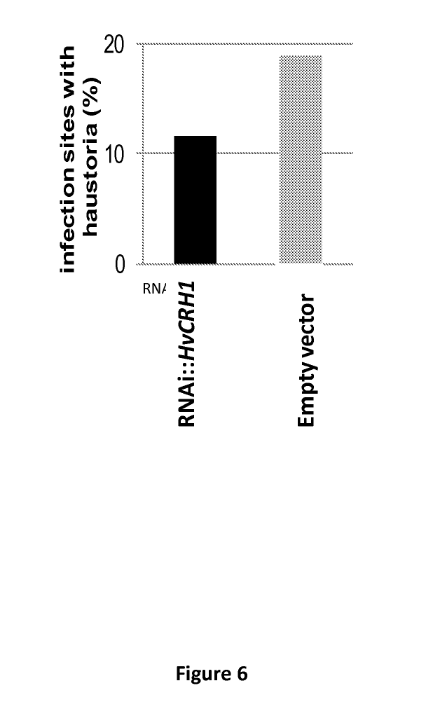

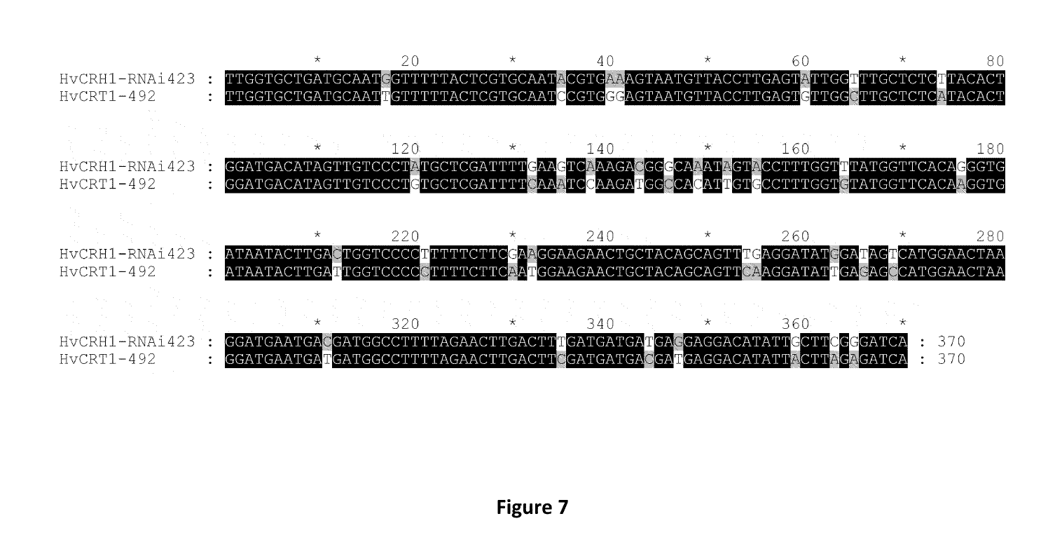

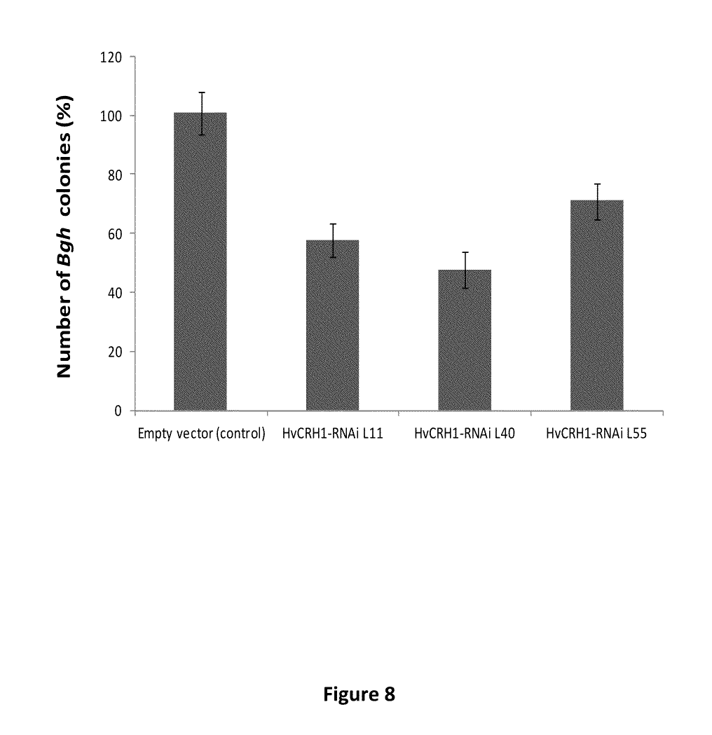

A second type of experiment was conducted to demonstrate the applicability of HvCRT1 modulation for improving disease resistance in a crop plant. In this set of experiments HvCRH1 expression was suppressed via RNAi-based silencing. Barley plants (Sultan5 bearing Mla12) were grown in a growth chamber at 18° C. with 60% relative humidity and a photoperiod of 16 h (60 μmol photons m⁻² s⁻¹). Segments of seven-day-old first leaves were shot with a 35S-HvCRH1-RNAi construct (p-AB 35S-RNAi ZeBaTA #423-3; FIG. 5, containing two inverted 35S promoters). Since HvCRH1-RNAi423 shares 336 nt of 370 nt with HvCRT1 and contains 4 regions of 100% identity with HvCRT1 of 20 nt or longer including one of 35 nt (FIG. 8), it should silence HvCRT1 as well as HvCRH1. This plasmid was co-bombarded with plasmid pGY1-GFP. As a control, an empty vector together with pGY1-GFP was used. After 24 h, segments were inoculated with approx. 140 conidia mm⁻² of Blumeriagraminis f. sp. hordei, race A6. Penetration frequencies on transformed cells were assessed using fluorescence (GFP) and light microscopy. FIG. 6 shows the result of an experiment in which the number of GFP-fluorescing cells that were attacked by Bgh-A6 allowed successful penetration (development of mature or immature haustoria). Similar results were obtained in 5 replicate experiments using either the cv. Sultan5 or the Pallas backcross line BCPallas-Mla12 as plant host. For each individual experiment, at least 150 interaction sites were evaluated. Stomata cells and stomata guard cells were excluded from the evaluation.

Constructs used in the experiments:
A: Plasmid p-AB 35S-RNAi ZeBaTA #423-3 together with pGY1-GFP.
B: Control: plasmids p-AB 35S-GUSi containing a fragment of uidA gene together with pGY1-GFP.

The number of successfully penetrated cells is reduced by 33% when cells were treated with the HvCRH1-silencing construct (35S-HvCRH1, #423-3). The result shows that silencing of the CRH1 genes leads to strongly reduced fungal penetration rates and thus improves resistance of those plants to powdery mildew. Please note that it is well established that a reduction in the frequency of successful penetration strongly correlates with enhanced disease resistance (see also e.g. Hückelhoven et al. 2003). It should also be noted that this enhancement is in addition to the already high level of resistance provided by the disease resistant gene Mla12.

The results obtained using the transient expression/silencing assay above were confirmed using stably transformed barley. Transgenic barley (Hordeumvulgare cv. Golden Promise) were generated using two transformation vectors (i) the binary vector pLH6000 (DNA Cloning Service, Hamburg, Germany; empty vector control), and (ii) the RNA interference vector pLH6000 UBI::CRH1::UBI (for silencing HvCRH1/HvCRT1 expression). Both of the vectors was introduced into the Agrobacterium strain AGL1 (Lazo et al., 1991) by electroporation (E. coli Pulser, Bio-Rad, Munich, Germany). Agrobacterium-mediated transformation, selection, and regeneration of roots were performed as described by Imani et al. 2011.

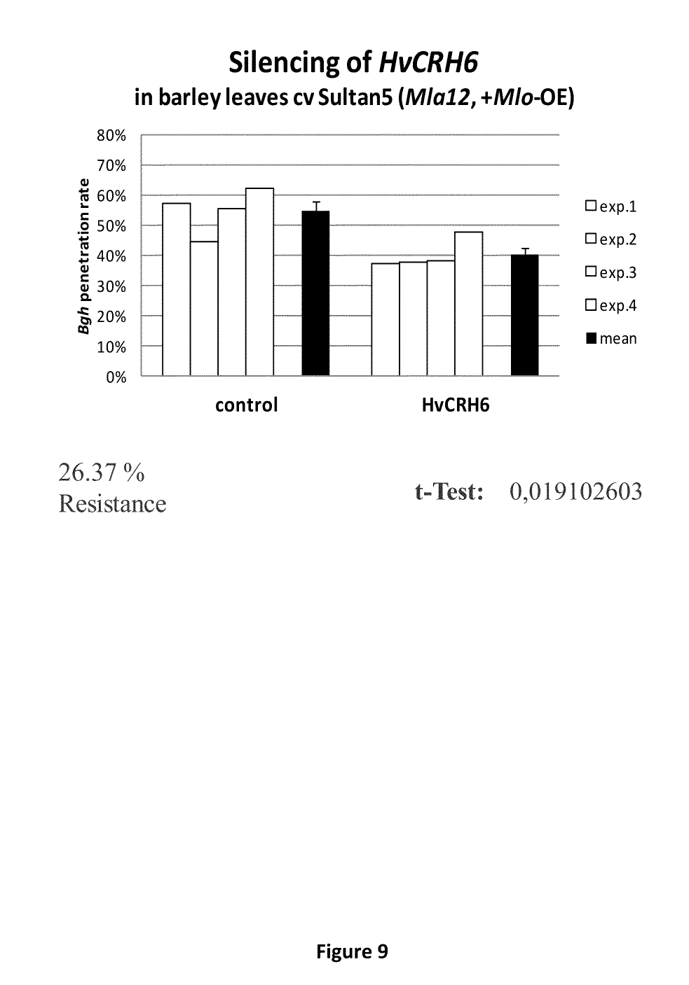

Multiple, independent transgenic lines were generated for both HvCRH1-RNAi and HvCRH6-RNAi. Many plants from each of these knockdown lines were inoculated with Blumeriagraminis together with control plants that were transformed with an empty vector. Basal resistance to Blumeriagraminis was enhanced in both HvCRH1-silenced plants (FIG. 8) and in HvCRH6-silenced plants (FIG. 9). Note that the differences in the levels of enhanced basal resistance among the HvCRH1-silenced lines (FIG. 8) did not correlate with the difference in levels of silencing of HvCRH1 since HvCRH1 was knocked down to similar levels in all three lines. This discrepancy may reflect (or be due to) the compensatory up regulation of other family members that was observed.

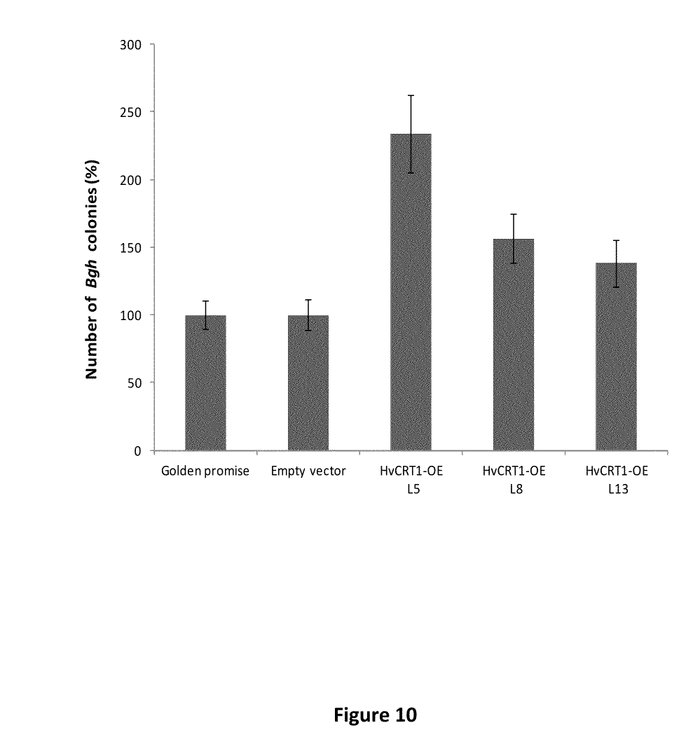

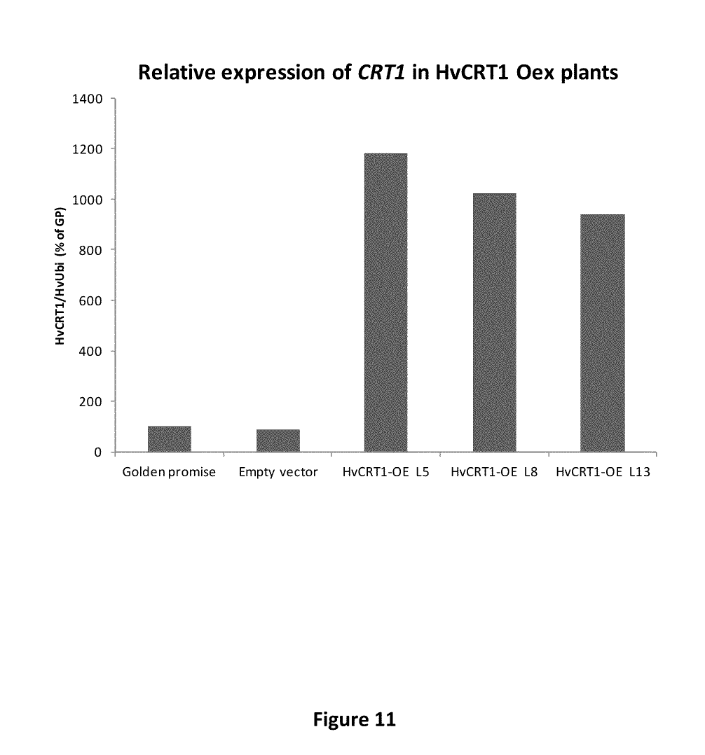

The HvCRH1-RNAi knockdown plants were also assessed for resistance to Fusarium graminearum. Basal resistance as measured by disease severity was enhanced in the coleoptile and particularly in leaves (FIG. 10). Moreover, growth of both roots and shoots was enhanced in the knockdown transgenic plants infected with this fungal pathogen (FIG. 11).

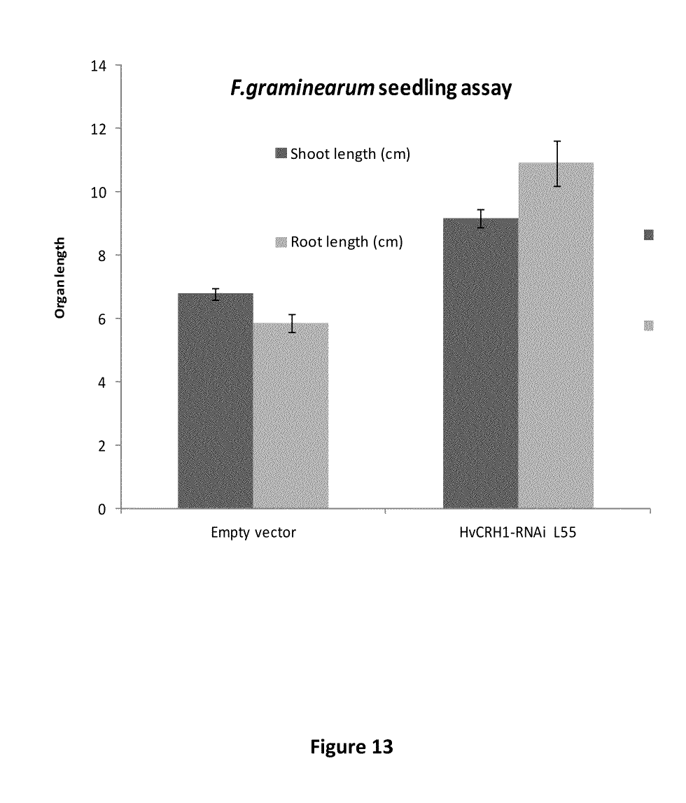

Stable transgenic barley over expressing HvCRT1 under the strong cauliflower mosaic virus (CaMV) 35S promoter were also constructed and assessed for the resistance to B. graminis and level of over expressed suppressed basal resistance (FIG. 12). The amount of suppression correlated with the amount of overexpression (FIG. 13).

### Example 2

### Assessment of Function of CRT1 Gene Family in Dicot Crops in Response to Pathogens

Tomato (Solanum lycopersycum) and its close relative potato (Solanum tuberosum) each contain CRT1 and five homologs—three in Glade II and two in Glade III (FIG. 1). Both are important crop species, with potato being the 3^{rd} most important crop worldwide after rice and maize. CRT1's role in resistance to the devastating late blight disease caused by Phytothphora infestans was assessed in RNAi silenced transgenic plants or plants over expressing CRT1 under the estradiol-inducible promoter.

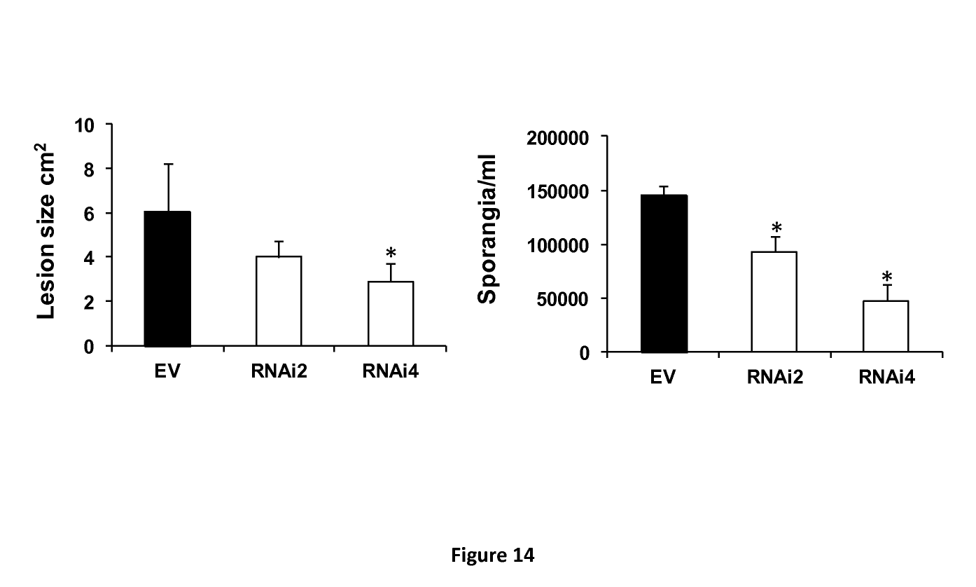

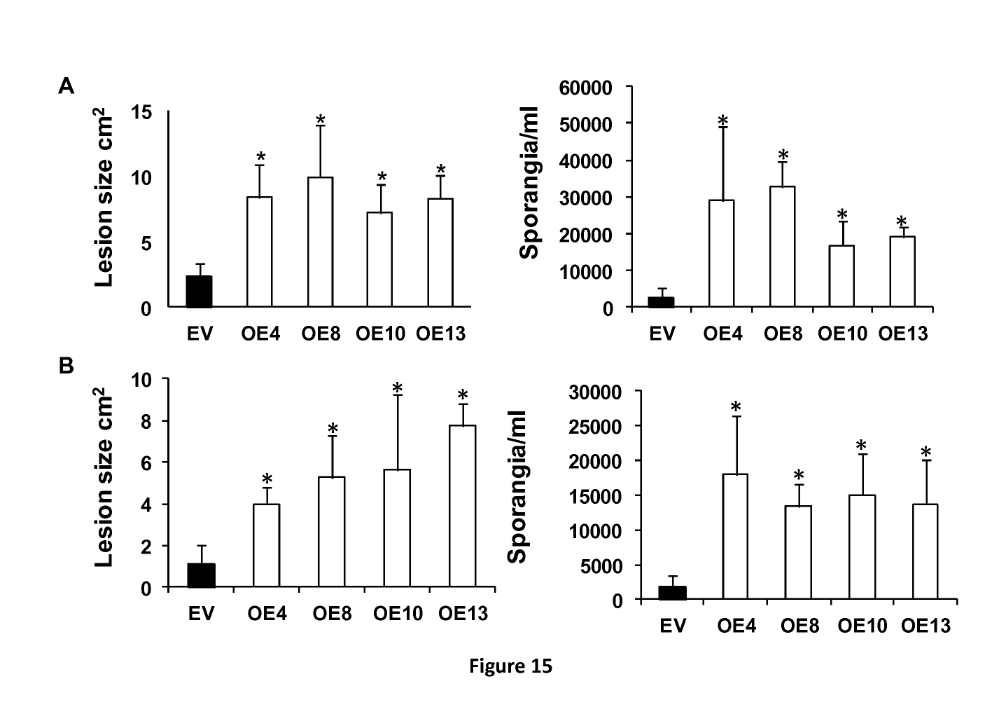

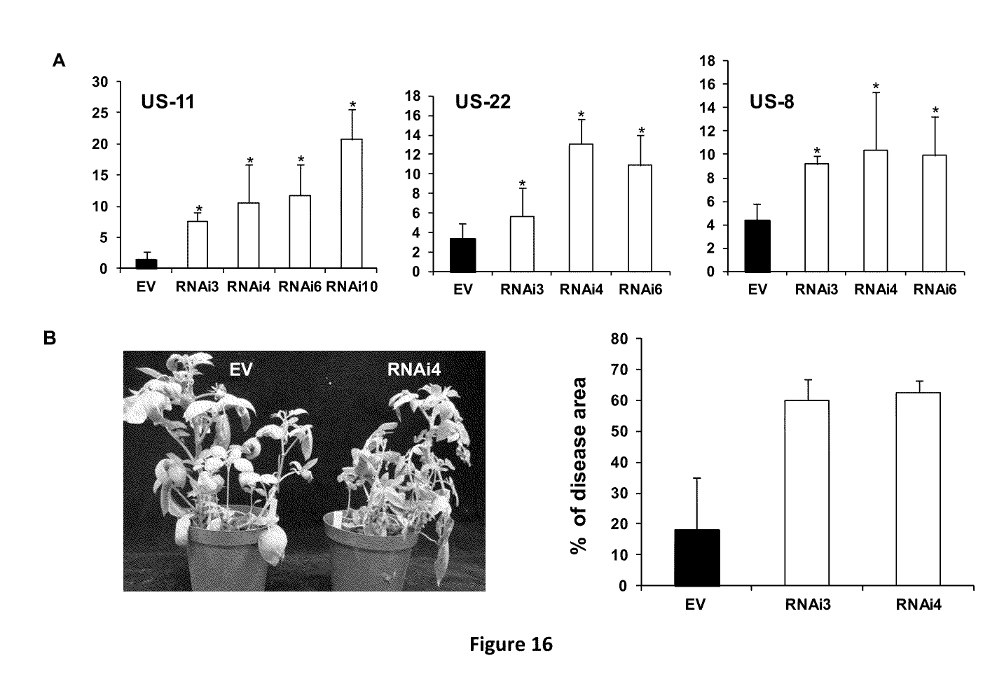

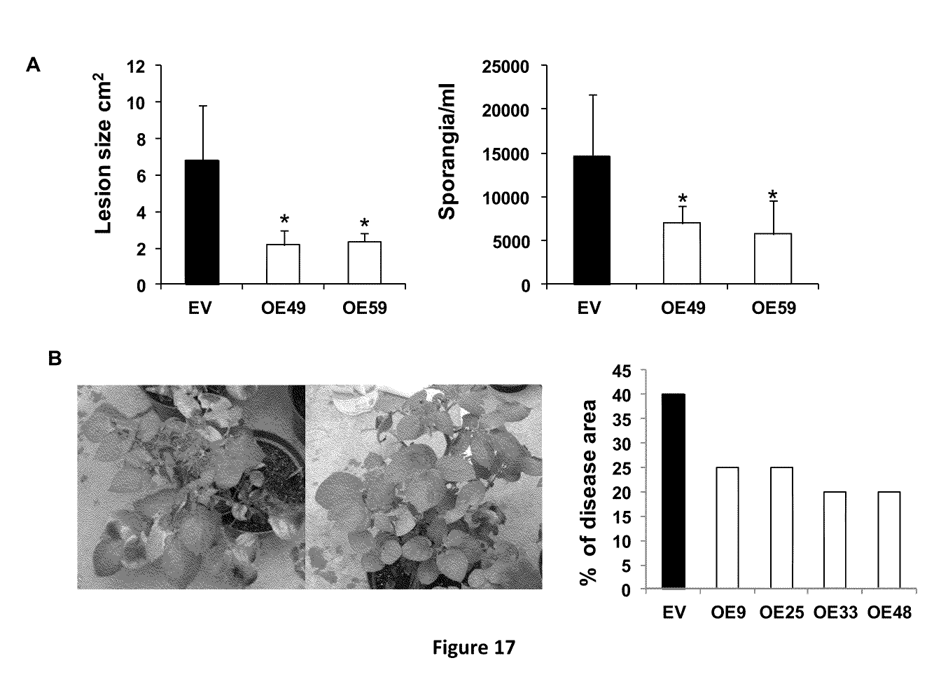

In tomato, silencing of SlCRT1 enhanced basal resistance to P. infestans (FIG. 14), while its over expression suppressed basal resistance (FIG. 15). In contrast, in potato silencing of StCRT1 suppressed basal resistance to this pathogen (FIG. 16) while its over expression enhanced basal resistance (FIG. 17). These results illustrate the species-specific nature of the effects on disease resistance of modulating expression/function of CRT1 family members.

### Example 3

### Genetically Engineered MORC Proteins and Peptides to Protect Plants from a Diverse Set of Bacterial, Fungal, Oomycete, and Viral Pathogens

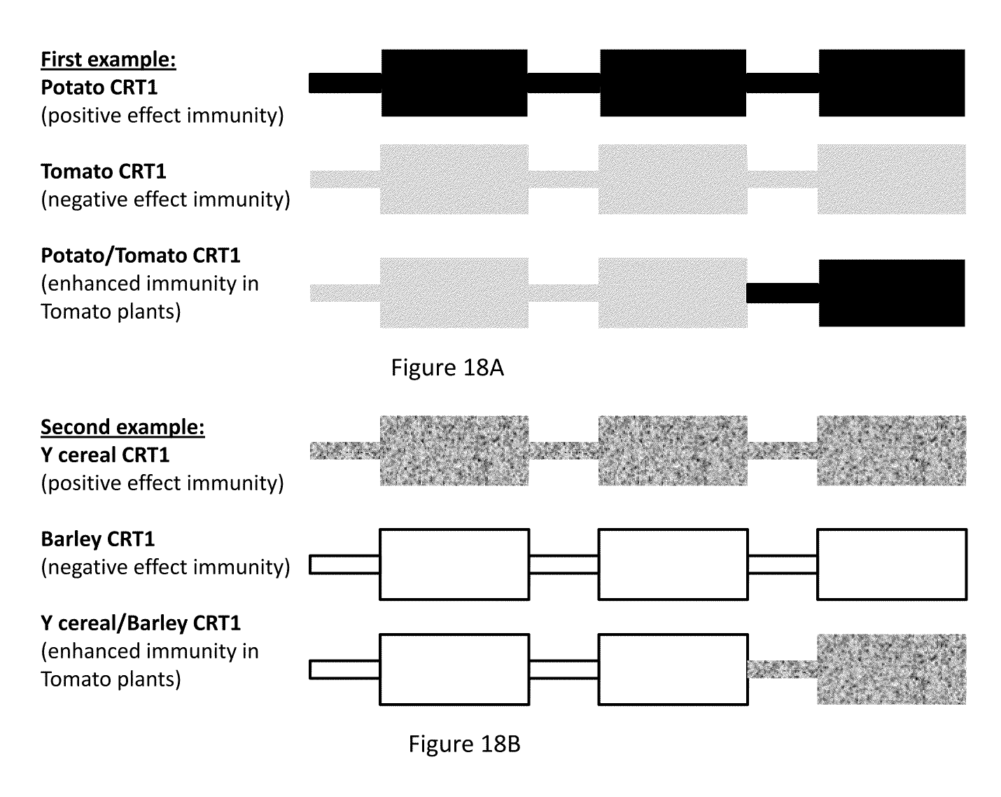

As described in the previous examples and as shown in the Table below, altering CRT1 expression has species specific effects on disease resistance. These differences appear to be due to differences in CRT1 proteins from different plants, e.g., differences in the 2^{nd} linker and CC domain. See FIG. 18.

TABLE

| | Arabidopsis | | Potato | | Tomato | | N. benthamiana | | Barley | |
|---|---|---|---|---|---|---|---|---|---|---|
| Resistance | Knock out | OE | RNAi | OE | RNAi | OE | VIGS | OE | RNAi | OE |
| PTI | [symbol] | [symbol] | [symbol] | [symbol] | [symbol] | [symbol] | [symbol] | NT | [symbol] | [symbol] |
| ETI | [symbol] | [symbol] | [symbol] * | NT | [symbol] | NT | NT | NT | [symbol] | [symbol] |

PTI PAMP-triggered (Basal) Immunity
ETI Effector triggered immunity (R gene-mediated resistance)
OE Transgenic plants overexpressing CATS
RNAi Transgenic plants suppressing CRT1 expression
VIGS Virus induced gene silencing
NT Not tested [symbol]
No effect
* Modest suppression

Our data show that the CRT1 protein (a MORC protein) modulates plant immunity to a diverse set of pathogens by playing a positive role in immunity in some species such as potato, while negatively affecting immunity in other species such as tomato and barley. Recent research has uncovered the parts of the protein that are responsible for this differential effect of CRT1 on immunity. Results of domain swapping experiments in which different parts of the proteins from potato and tomato were interchanged, demonstrated that a CRT1 that negatively influenced immunity could be converted to a hybrid that positively affected immunity (see below), thereby providing the potential to improve immunity by its over expression or ectopic expression. See FIG. 18. In view of these findings, the present inventors can now generate CRT1 molecules which influence disease resistance as desired in plants of interest.

### REFERENCES

Felsenstein J. (1985). Confidence limits on phylogenies: An approach using the bootstrap. Evolution 39:783-791.
Hückelhoven R, Dechert C, Kogel K H (2003) Over-expression of barley BAX Inhibitor-1 induces enhanced accessibility to Blumeriagraminis and breakdown of mlo-mediated penetration resistance in barley. Proc. Nat. Acad. Sci. USA 100, 5555-5560.
Imani J, Li L, Schafer P, Kogel K H (2011) STARTS—A stable root transformation system for rapid functional analyses of proteins of the monocot model plant barley. Plant Journal DOI: 10.1111/j.1365−313X.2011.04620.x
Jansen C, von Wettstein D, Schafer W, Kogel K H, Felk A, and Maier, F J (2005) Infection patterns in barley and wheat spikes inoculated with wild-type and trichodiene synthase gene disrupted Fusariumgraminearum. PNAS 102, 16892-16897
Jones D. T., Taylor W. R., and Thornton J. M. (1992). The rapid generation of mutation data matrices from protein sequences. Computer Applications in the Biosciences 8: 275-282.
Kang, H.-G., Kuhl, J. C., Kachroo, P., and Klessig, D. F. (2008) CRT1, an Arabidopsis ATPase that interacts with diverse resistance proteins and modulates disease resistance to Turnip Crinkle Virus. Cell Host & Microbe. 3:48-57.
Kang, H.-G., Oh, C.-S., Sato, M., Katagiri, F., Glazebrook, J., Takahashi, H., Kachroo, P., Martin, G., and Klessig, D. F. (2010) Endosome-associated CRT1 functions early in resistance gene-mediated defense signaling in Arabidopsis and Tobacco. Plant Cell, 22:918-936.
Lazo G R, Stein P A, Ludwig R A. (1991) A DNA transformation-competent Arabidopsis genomic library in Agrobacterium. Biotechnology (N Y). 9, 963-7.
Schweizer P, Pokorny J, Abderhalden O, Dudler R (1999) A transient assay system for the functional assessment of defense related genes in wheat. Mol Plant-Microbe Interact 12: 647-654
Schweizer P, Pokorny J, Schulze-Lefert P, Dudler R (2000) Technical advance. Double-stranded RNA interferes with gene function at the single-cell level in cereals. Plant J 24: 895-903
Sneath P H A & Sokal R R (1973) Numerical Taxonomy. Freeman, San Francisco.
Tamura K., Dudley J., Nei M., and Kumar S. (2007). MEGA4: Molecular Evolutionary Genetics Analysis (MEGA) software version 4.0. Molecular Biology and Evolution 24:1596-1599.
McCallum C M, Comai L, Greene E A, Henikoff S. (2000) Targeted screening for induced mutations. Nat Biotechnol. 18:455-7.
McCallum C M, Comai L, Greene E A, Henikoff S. (2000) Targeting induced local lesions IN genomes (TILLING) for plant functional genomics. Plant Physiol. 123:439-42.
Slade A J, Fuerstenberg S I, Loeffler D, Steine M N, Facciotti D. (2005) A reverse genetic, nontransgenic approach to wheat crop improvement by TILLING. Nat Biotechnol. 23:75-81.
Erik Vollbrecht, Jon Duvick, Justin P. Schares, Kevin R. Ahern, PrasitDeewatthanawong, Ling Xu, Liza J. Conrad, Kazuhiro Kikuchi, Tammy A. Kubinec, Bradford D. Hall, Rebecca Weeks, Erica Unger-Wallace, Michael Muszynski, Volker P. Brendel, and Thomas P. Brutnell (2010) Genome-Wide Distribution of Transposed Dissociation Elements in Maize. Plant Cell 22: 1667-1685.
Zuckerkandl E & Pauling L (1965) Evolutionary divergence and convergence in proteins, pp. 97-166 in Evolving Genes and Proteins, edited by V. Bryson and H. J. Vogel. Academic Press, New York.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for producing a plant exhibiting increased pathogen resistance comprising,
a) introducing a nucleic acid construct encoding RNAi specific for silencing of CRT1 and its closely related homologs into a plant cell, said RNAi effectively inhibiting CRT1 or CRT1 homolog gene expression in said plant cell, said cell exhibiting increased pathogen resistance when compared to wild type plant cells lacking said RNAi.

2. The method of claim 1, wherein said RNAi is under the control of a constitutive promoter.

3. The method of claim 1, wherein said RNAi is under the control of an inducible promoter.

4. The method of claim 3, wherein said promoter is induced upon infection with a pathogen.

5. A plant produced from the plant cell obtained by any one of claim 1, 2, 3, or 4.

6. The plant of claim 5 which is barley or tomato.

7. A nucleic acid construct encoding a CRT1 or CRT1 homolog specific RNAi which is effective to down modulate expression of said CRT1 or CRT1 homolog in a plant of interest.

8. A plant cell comprising the construct of claim 7.

9. A method for producing plants exhibiting increased pathogen resistance using the TILLNG method, comprising
a) treating plant seeds with an effective amount of an agent effective to introduce mutations into the plant genome,
b) screening the progeny plants for the presence of lesions in a CRT1 or CRT1 homolog gene, the lesions resulting in reduced production of functional CRT1 or CRT1 homolog protein,
c) testing the plants of step b) for enhanced resistance to pathogens compared to untreated plants.

10. A method of producing plants which exhibit enhanced pathogen resistance comprising crossing plants identified using the method of claim 9, such that progeny plants resulting from said cross exhibit enhanced pathogen resistance.

11. A method for producing plants exhibiting increased pathogen resistance using a transposable element system, comprising
a) crossing, by breeding, a plant, the cells of which harbor transposon elements in their genomes near a CRT1 or CRT1 homolog gene with a plant comprising a nucleic acid which encodes an active transposase, the transposase catalyzing in the progeny plants, transposition of the transposon element into the surrounding DNA including the CRT1 or CRT1 homolog gene,
b) screening plants so treated for the presence of lesions in said gene, the lesions being correlated with reduced production of functional CRT1 or CRT1 homolog protein,
c) testing the plants of step b) for enhanced resistance to pathogens compared to untreated plants.

12. The method of claim 11, wherein said transposable element system is the Ac/Ds system.

13. A plant produced by the method of claim 9, 10 or 11.

14. The plant of claim 13, which is barley.

15. A method for producing a plant exhibiting increased pathogen resistance comprising, introducing a nucleic acid construct encoding CRT1 or its closely related homologs into a plant cell, thereby over-expressing CRT1, overexpression of CRT1 in said cell being correlated with increased pathogen resistance when compared to wild type plant cells lacking said construct, with the proviso said plant is not Arabidopsis.

16. The method of claim 15, wherein said nucleic acid is under the control of a constitutive promoter.

17. The method of claim 15, wherein said nucleic acid is under the control of an inducible promoter.

18. The method of claim 17, wherein said promoter is induced upon infection with a pathogen.

19. A plant produced from the plant cell obtained from the method of any one of claim 15, 16, 17, or 18.

20. The method of claim 19, wherein said plant is potato.

21. The method of any one of claim 1, 9, 10, 11 or 15, wherein said CRT1 homolog is selected from the group consisting of CRH1, CRH2, CRH3, CRH4, CRH5 and CRH6.

22. A nucleic acid encoding a chimeric CRT1 protein which upon expression in a plant alters a disease resistance phenotype in said plant.

23. The nucleic acid of claim 22, comprising CRT1 domains from potato and tomato.

24. The nucleic acid of claim 22, comprising CRT1 domains from a first cereal and barley.

25. A protein encoded by the nucleic acids of claim 23 or claim 24.

26. A plant comprising the nucleic acid of claim 23 or 24.


SEQUENCE LISTING (9 sequences)

SEQ ID NO: 1 - DNA, 26 bp, Artificial Sequence
  Primer
cccgggaaaccctaaccttccaatgc

SEQ ID NO: 2 - DNA, 27 bp, Artificial Sequence
  Primer
aagctttcacatgtatgggagctgctg

SEQ ID NO: 3 - DNA, 20 bp, Artificial Sequence
  Primer
gagacttggtgctgatgcaa

SEQ ID NO: 4 - DNA, 20 bp, Artificial Sequence
  Primer
ttttgaccttgatcccgaag

SEQ ID NO: 5 - DNA, 1846 bp, Artificial Sequence
  Synthetic Sequence
cccgggaaaccctaaccttccaatgccagcggcaatggccggcggcgatggcggcatgggcagtggccctcgatccctcgattgccgtagcttctggaaggccggcgcgttcgaggccccctccgccgccgcccgcgagttctacgacgtgctggagacaggggacttcgaccgcgcgcgggtgcacccgaagttcctgcacaccaacgcgacctcccacaagtgggcgttcggagctatagctgaacttcttgacaatgcagttgatgagatttgcaatggagccacattcataaaagtggataaaagcatcaatttaaaagacagtagcccaatgctggttttccaagacgatggaggaggaatggatcctgaaggtgtacggcaatgcataagtttaggattctcaaccaagaaatcaaagacaaccattggccagtatggaaatggctttaagacaagcacaatgagacttggtgctgatgcaattgtttttactcgtgcaatccgtgggagtaatgttaccttgagtgttggcttgctctcatacactttcttgaggagaacaatgaaggatgacatagttgtccctgtgctcgattttcaaatccaagatggccacattgtgcctttggtgtatggttcacaaggtgattgggatagtagcttgaagataatacttgattggtcccccttttcttcaatggaagaactgctacagcagttcaaggatattgagagccatggaactaaggtggtgatatatgatctatggatgaatgatgatggccttttagaacttgacttcgatgatgacgatgaggacatattacttagagatcaagctaaagctactgcggggacgacaaagatccaaaaagaaattattgagcaacatatatcccacagactcagattctctttgcgcgcgtatacttccatcctttatcttaagaaatatgcgaacttccaaattatattaaggggaaaagtggttgaacatataagtgttgcccatgatctgaagtttaagaaagtatttacttacaagcctcaagttacgcatgattctcaagtggtctcagtgaaggtagatgttggatttgccaaggaggcaccagttttgggcatttttgggatgaatgtctaccataaaaatcgactaataatgcccttctggaaggttcttcaggaaggatctagcagagggaggagtgttgtaggtgtacttgaggcaaattttattgaaccggcacatgacaaacaggattttgagaggactccactattcattcgtctggaaactaaacttagacaaattatcattgagtactggaaaaacaactgtcatttgataggttaccagccaatgaatccacaattaaaaacacagtataaagctgccaaagctccaggtggtcctggacatcagtttcagaagaaatcgtctactgctcagaggattggagcacattcatcaaatttgctaccggaaacatatgatgacacagcagtctttggattgtcagctaatggtgcaggttctggtttgcaattttctggccgagcacaagaaaaaagtacaaattcagcaggcttggaagaggatctagtcaatattgcctctgatggtgaacttgatccgaatgtcattgagaagctgagtgatgaaaacatttctctgttcacaaggcgtgaggagcttaaacaacgagatacacaattgaagcagacgattttggagttggagcatgaactagaggaaacaaaaaggaaatgctctcagctttctactgagctgcaggtgcggaagagccagcagcagctcccatacatgtgaaagctt

SEQ ID NO: 6 - protein, 605 aa, Hordeumvulgare
MPAAMAGGDGGMGSGPRSLDCRSFWKAGAFEAPSAAAREFYDVLETGDFDRARVHPKFLHTNATSHKWAFGAIAELLDNAVDEICNGATFIKVDKSINLKDSSPMLVFQDDGGGMDPEGVRQCISLGFSTKKSKTTIGQYGNGFKTSTMRLGADAIVFTRAIRGSNVTLSVGLLSYTFLRRTMKDDIVVPVLDFQIQDGHIVPLVYGSQGDWDSSLKIILDWSPFSSMEELLQQFKDIESHGTKVVIYDLWMNDDGLLELDFDDDDEDILLRDQAKATAGTTKIQKEIIEQHISHRLRFSLRAYTSILYLKKYANFQIILRGKVVEHISVAHDLKFKKVFTYKPQVTHDSQVVSVKVDVGFAKEAPVLGIFGMNVYHKNRLIMPFWKVLQEGSSRGRSVVGVLEANFIEPAHDKQDFERTPLFIRLETKLRQIIIEYWKNNCHLIGYQPMNPQLKTQYKAAKAPGGPGHQFQKKSSTAQRIGAHSSNLLPETYDDTAVFGLSANGAGSGLQFSGRAQEKSTNSAGLEEDLVNIASDGELDPNVIEKLSDENISLFTRREELKQRDTQLKQTILELEHELEETKRKCSQLSTELQVRKSQQQLPYM

SEQ ID NO: 7 - DNA, 13 bp, Artificial Sequence
  SfiI site
ggccnnnnnggcc

SEQ ID NO: 8 - DNA, 310 bp, Artificial Sequence
  HvCRH1-RNAi423
ttggtgctgatgcaatggtttttactcgtgcaatacgtgaaagtaatgttaccttgagtattggtttgctctcttacactggatgacatagttgtccctatgctcgattttgaagtcaaagacgggcaaatagtacctttggtttatggttcacagggtgataatacttgactggtcccctttttcttcgaaggaagaactgctacagcagtttgaggatatggatagtcatggaactaaggatgaatgacgatggccttttagaacttgactttgatgatgatgaggaggacatattgcttcgggatca

SEQ ID NO: 9 - DNA, 310 bp, Artificial Sequence
  HvCRT1-492
ttggtgctgatgcaattgtttttactcgtgcaatccgtgggagtaatgttaccttgagtgttggcttgctctcatacactggatgacatagttgtccctgtgctcgattttcaaatccaagatggccacattgtgcctttggtgtatggttcacaaggtgataatacttgattggtcccccttttcttcaatggaagaactgctacagcagttcaaggatattgagagccatggaactaaggatgaatgatgatggccttttagaacttgacttcgatgatgacgatgaggacatattacttagagatca